(19) United States
(12) Patent Application Publication
(10) Pub. No.: US 2019/0021702 A1
(43) Pub. Date: Jan. 24, 2019

(54) EXTRACTION AND ANALYSIS DEVICE, IN PARTICULAR FOR SYNOVIAL FLUID

(71) Applicant: EON MEDICA S.R.L., Monza (IT)

(72) Inventors: Emanuela Maria CECCA, Monza (IT); Renza CULTRERI, Monza (IT); Tommaso BRENICCI, Monza (IT)

(73) Assignee: EON MEDICA S.R.L., Monza (IT)

(21) Appl. No.: 15/755,762

(22) PCT Filed: Aug. 31, 2015
(86) PCT No.: PCT/IT2015/000215

§ 371 Date: Feb. 27, 2018

(51) Int. Cl. A61B 10/00; A61B 5/00

(52) U.S. Cl. CPC A61B 10/0045; A61B 2010/045; A61B 5/4528

(57) ABSTRACT

The present invention concerns a device for extraction and analysis (1) of anatomical tissues, such as synovial liquid and the like, comprising an extraction line (2, 21, 211, 212) of said tissue to be analyzed, having a needle (22), for the withdrawal of said tissue to be analyzed, an extraction duct (21, 211, 212), an extraction syringe (24), having an inlet/outlet mouth connected fluid-dynamically to said needle (22) by means of said extraction duct (21, 211, 212), and an analysis device (3), fluid-dynamically connected to said inlet/outlet mouth of said extraction syringe (24) by means of said extraction duct (21, 211, 212), said analysis device (32) having one or more receptacles (321, 322, 323), in each of which a respective reagent is arranged, and reading means (33), for detecting possible alterations of said reagents.

The present invention relates to an extraction and analysis device, in particular for synovial fluid.

More specifically, the invention relates to a device for withdrawing and in-line analysis, studied and realized in particular to allow real-time high reliability analysis of withdrawn synovial fluid, so as to have a first response for possible infection, usable in field of surgical interventions, such as for the removal of prostheses and the like, which can also be used in ambulatory or in any other situation where it is necessary to have real time diagnostic clinical results.

In the following, the description will be directed to the withdrawal and the analysis of synovial fluid, but it is clear that the same should not be considered limited to this specific use.

As it is well known at present an accurate and efficient diagnosis of periprosthetic joint infection (PJI) remains one of the most felt needs for orthopedic and microbiologists surgeons.

Several tests are commercially available for the diagnosis of articular periprosthetic infection. In recent years, many studies have been carried out to identify new markers or reagents that can contribute to a more accurate diagnosis of osteoarticular infection.

Among the potential markers or reagents of osteoarticular infection only a few have certain characteristics that make them suitable for the use in the clinical diagnostics, making as they are easily detectable in the synovial fluid through automated or easy to carry out methods.

In particular, leukocyte esterase (Leukocyte Esterase—or “LE”) test is easy to carry out and easy to apply in a complex diagnostic process, which is the diagnosis of bone and joint infections.

The leukocyte esterase, also known as neutrophil elastase, or granulocyte elastase, is an enzyme synthesized by white blood cells, particularly by activated neutrophils, often found within infected body fluids.

The esterases are enzymes belonging to the class of hydrolases, which catalyze the hydrolysis reaction of the ester bond. In particular, the leukocyte esterase has the function to degrade proteins that act as a support to the connective tissue matrix.

The secretion of the enzyme leukocyte esterase by neutrophils retrieved by the infection condition, allows checking with a simple enzymatic colorimetric test the presence of an infection. This test involves, in fact, a change of the color of the pad, which depends by a hydrolytic reaction that occurs when the leukocyte esterase enzyme contacts with the chemical substances on the test pad.

The presence of granulocytic leukocytes is confirmed on the basis of the color gradation.

Furthermore, it was previously assumed (see in particular the article “Preliminary Results of a New Test for Rapid Diagnosis of Septic Arthritis with Use of Leukocyte Esterase and Glucose Reagent Strips”, Mohamed Omar, Max Ettinger, Moritz Reichling, Maximilian Petri, Ralf Lichtinghagen, Daniel Guenther, Eduardo M. Suero, Michael Jagodzinski, and Christian Krettek, The Journal Of Bone And Joint Surgery, Incorporated), that the combined analysis of leukocyte esterase and glucose can improve the diagnosis of septic arthritis in the native synovial fluid, because the concentrations of leukocyte esterase are high at the site of infection, due to the increased secretion of this enzyme by neutrophils in case of inflammatory processes, while the glucose concentration is rather reduced in the case of infectious process, due to the bacterial metabolism.

In known extraction and diagnosis systems of synovial liquid a problem is that of transferring the same into vacutainers or tubes in general, in which it is highly probable contamination and loss of fluid.

In addition, a well-known problem in the field is that of allowing, at the same time, a sterile collection of the synovial liquid in a vacutainer, for it to be analyzed at a later time in the laboratory and a first test, which in real-time allows to provide both an indication of the presence of inflammation of the withdrawn synovial fluid, and a first indication of the degree of the infection. Naturally, such effects are to be obtained in a totally sterile way, avoiding any external contamination.

In light of the above, it is, therefore, object of the present invention to provide a device for real-time analysis of synovial fluid, so as to obtain a reliable diagnosis immediately.

It is also object of the present invention, to enable the use of the entire synovial liquid extracted from the patient, without losses in line and without contamination with the outside environment between the extraction of the same and the run of the test.

It is therefore specific object of the present invention device for extraction and analysis of anatomical tissues, such as synovial liquid and the like, comprising an extraction line of said tissue to be analyzed, having a needle, for the withdrawal of said tissue to be analyzed, an extraction duct, an extraction syringe, having an inlet/outlet mouth connected fluid-dynamically to said needle by means of said extraction duct, and an analysis device, fluid-dynamically connected to said inlet/outlet mouth of said extraction syringe by means of said extraction duct, said analysis device having one or more receptacles, in each of which a respective reagent is arranged, and reading means, for detecting possible alterations of said reagents.

Always according to the invention, said analysis unit could comprise a first receptacle, into which a first reagent is placed, a second receptacle, into which a the second reagent is placed, and a third receptacle, into which a third reagent is placed.

Still according to the invention, said first reagent could be esterases, said second reagent is CRP (C-Reactive Protein) and said third reagent is glucose.

Advantageously according to the invention, said reading means could comprise a reading scale, capable to show the color variations of said reagents, allowing to detect the degree of infection of said anatomical tissue, as synovial liquid and the like.

Further according to the invention, said extraction duct of said extraction line could comprise a first three-way valve, having a first port, connected to said withdrawal needle, a second port, connected to said inlet/outlet mouth of said extraction syringe, and a third port connectable to said analysis device, each of said ports being selectively opened or closed independently of the other.

Always according to the invention, said extraction line could comprise air filtering means, in their turn comprising an air filter and a respective three-way valve, having three ports selectively openable or closable independently of the other, one of said three ports being connected to said inlet/outlet mouth of said extraction syringe, and another of said three ports being connected to said air filter.

Still according to the invention, said device could comprise an anatomical tissues collecting line, having a vacutainer, fluid-dynamically connected to said inlet/outlet mouth of said suction syringe.

Advantageously according to the invention, said collecting line could comprise a suction syringe having an inlet/outlet mouth fluid-dynamically connected to said inlet/outlet mouth of said extraction syringe.

Always according to the invention, said collecting line could comprise a second three-way valve, having a first port, fluid-dynamically connected to said third port of said first three-way valve along said extraction duct of said extraction line, a second port fluid-dynamically connected to said inlet/outlet mouth of said suction syringe, and a third port, fluid-dynamically connected to said analysis device, each of said ports being selectively opened or closed independently of the other.

Still according to the invention, said collecting line could comprise filtering means comprising an air filter and a respective three-way valve, having three ports selectively openable or closable independently of the other, one of said three ports being connected to said inlet/outlet mouth of said suction syringe, and another one of said three ports being connected to said air filter.

Advantageously according to the invention, said device could comprise plasma filtering means, adapted to filter the plasma from the said tissue to be analyzed, arranged between said inlet/outlet mouth of said extraction syringe and said analysis device.

Further according to the invention, said plasma filtering means comprise a filtering syringe, having an inlet/outlet mouth, a third three-way valve, having a first port, fluid-dynamically connected to said inlet/outlet mouth of said extraction syringe, a second port, to which the inlet/outlet mouth of said filtering syringe is connected, and a third port, fluid-dynamically and selectively connected to said analysis device.

Always according to the invention, said extraction line comprise a first duct, which connects said withdrawal needle and the analysis unit, said first three-way valve being arranged along said first duct, and a second duct, which connects the extraction syringe to said first duct, a four-way valve having a first port fluid-dynamically connected to said withdrawal needle, a second port fluid-dynamically connectable to said vacutainer, a third port fluid-dynamically connectable to said extraction syringe and a fourth port, each of said ports being selectively opened or closed independently of the other, and said filtering means could comprise a filtering line, fluid-dynamically connected between said filtering syringe and said analysis unit, and a further three-way valve, connected to said fourth port of said four-way valve.

Still according to the invention, said device could comprise closing means, as a clamp and the like, arranged in one or more of said ports of said four-way valve, said closing means being preferably arranged on said first, second and fourth ports.

Advantageously according to the invention, said four-way valve comprises a three-way valve, whose ports are selectively opened or closed independently of the other, and a fourth port.

Further according to the invention, said filtering means comprise lysing, preloaded within said filtering syringe.

Always according to the invention, said filtering means comprise a filter for blood contamination interposed between said inlet/outlet mouth of said extraction syringe and said inlet/outlet mouth of said filtering syringe.

The present invention will be now described, for illustrative but not limitative purposes, according to its preferred embodiments, with particular reference to the figures of the enclosed drawings, wherein:

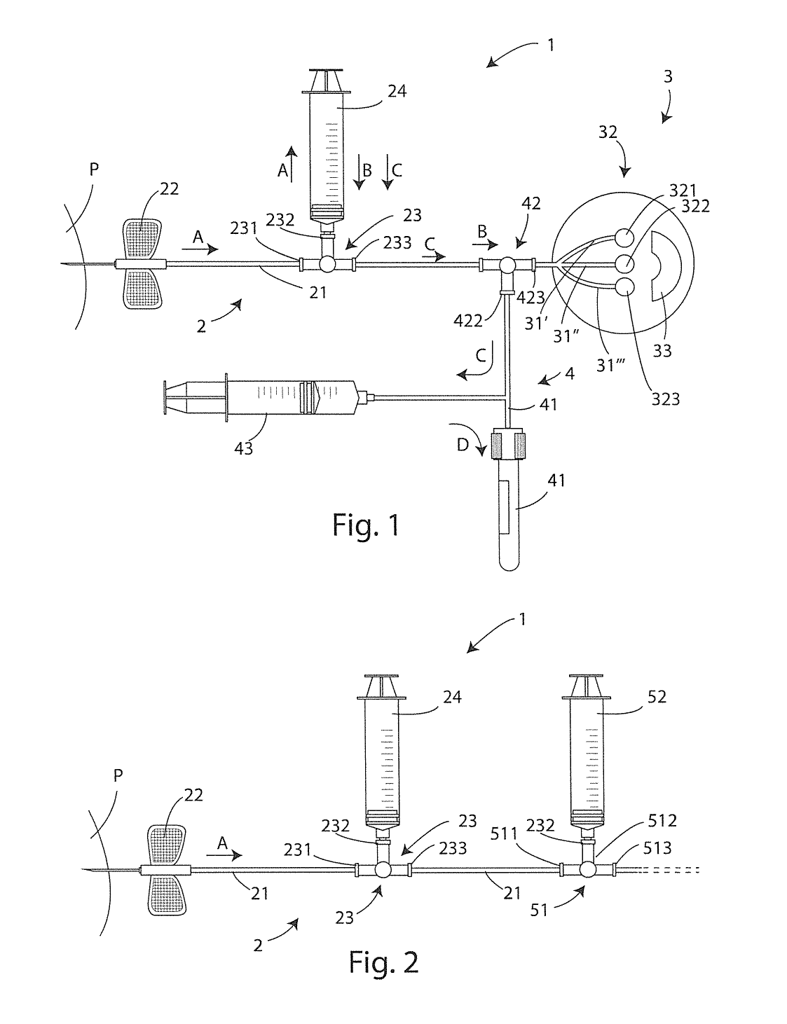

FIG. 1 shows the scheme of a first embodiment of an extraction and analysis device for synovial liquid according to the present invention;

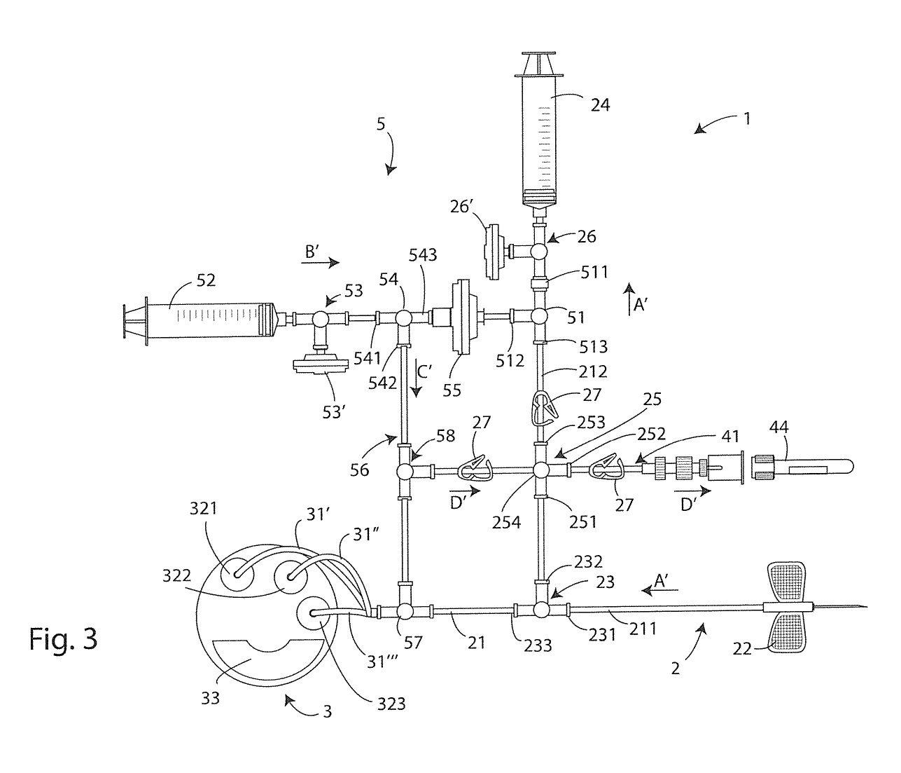

FIG. 2 shows a part of a second embodiment of an extraction and analysis device according to the present invention; and

FIG. 3 shows the scheme of a third embodiment of an extraction and analysis device for synovial liquid according to the present invention.

In the various figures, similar parts will be indicated by the same reference numbers.

Referring to FIG. 1, an extraction and analysis device 1 for synovial liquid according to the present invention is seen. Naturally, the extraction and analysis device 1 can also be used for other anatomical tissues in liquid form.

The extraction and analysis device 1 essentially comprises a synovial liquid extraction line 2, an analysis unit 3, and a collecting line of the synovial liquid 4.

Said extraction line 2 comprises a extraction duct 21, a withdrawn needle 22, connected to said extraction duct 21, a first three-way valve 23, arranged along said extraction duct 21, and an extraction syringe 24 to allow the extraction of the synovial liquid from a patient P.

Said first three-way valve 23 has three ports, respectively a first port 231, fluid-dynamically connected, i.e. by means of the duct 21, with said withdrawn needle 22, a second port 232, connected with said extraction syringe 24, and a third port 233, whose connection is better defined in the following.

Ports 231, 232 and 233 of said three-way valve 23 are all selectively openable or closable independently from the other and allow the passage in input or output of a fluid, which, as said, in the present case is synovial fluid.

Said analysis unit 3 comprises three ducts 31′, 31″ and 31′″, all connected to the end of said extraction duct 21, opposite to the one to which said withdrawn needle 22 is connected.

Said analysis unit 3 also includes an analysis device 32, having three receptacles 321, 322 and 323, in which respectively in three different reagents are arranged. In particular, esterase is placed in said first receptacle 321, CRP (C-Reactive Protein) is placed in said second receptacle 322 and Glucose is placed in said third receptacle 323.

The analysis device 32 also comprises a reading scale 33, the operation of which will be better described below.

The collecting line of the synovial fluid 4 comprises a collecting duct 41, having one end connected to a port of a second three-way valve 42, arranged on said extraction duct 21, of the same type of said first three-way valve 23 and arranged downstream of the latter.

Said second three-way valve 42 has a first port 421, connected with the section of the extraction duct 21 connected to said third port 233 of said first three-way valve 23, a second port 422, connected to said collecting duct 41, and a third port 423, connected to the section of the extraction duct 21 connected to said analysis device 3. Said three-way valve 42 is therefore arranged along said extraction duct 21, interposed between said first three-way valve 23 and said analysis device 32.

Said synovial liquid collecting line 4 also includes a vacutainer 44, removably connected to one end of said collecting duct 41.

Said vacutainer 44 is intended for collecting synovial fluid. Said synovial liquid collecting line 4 comprises also, in the present embodiment a suction syringe 43, connected to said collecting duct 41.

The operation of the extraction and analysis device 1 described above is as follows.

Once the extraction needle 22 is inserted, for example in an area of which verifying the possible presence of an infection is necessary, the third port 233 of said first three-way valve 23 is closed. In this way, the connection of the section of the extraction duct 21 between said first three-way valve 23 to said analysis device 32 or to the second three-way valve 42, is interrupted.

The ports 231 and 232 of said first three-way valve 23 are open. In this way, by acting on the extraction syringe 24, it is possible to extract the synovial fluid, collecting it in the reservoir of the extraction syringe 24 itself. The synovial fluid follows, with reference to FIG. 1, the arrow A.

Subsequently, the first door 231 is closed and the third door 233 opened. In addition, the first 421 and the third 423 port of the second three-way valve 42 are open, while the second port 422 remains closed. In this way, by pressing the plunger of the extraction syringe 24, gradually transferring at least part of the synovial fluid collected in said analysis device 3 is possible, following the arrow B. In this way, the synovial fluid is distributed on the three ducts 31′, 31″ and 31′″ and on the three receptacles 321, 322 and 323, thus entering in contact with the three reagents. The reading scale 33 shows the chromatic variations of the three reagents, thus allowing to detect the degree of infection of the synovial fluid in real time. In this way, the surgeon or the health professional has a first clinical picture, to promptly intervene if necessary.

In particular, in the table 1 below the data for glucose, leukocyte esterase and CRP are shown, for two groups of patients, which do not differ significantly for sex or age. The first group includes 45 patients and they are aseptic; the second patients group B includes 15 patients and they are infected.

In patients group A, only one patient has had a positive result (shown as 1+) for leukocyte esterase, while in other patients the leukocyte esterase has shown results either negative (n=51) or present in traces (n=9).

In contrast, in patients with infection, the leukocyte esterase has been shown as negative in 2 patients, 1+ in 11 patients and 2+ in 8 patients. In group A, the glucose was negative in 4 patients, detected in traces, 1+, 2+, respectively for 14, 37, 6 patients, while, in the test group B, glucose was negative in 10 patients, present in traces in 6 patients and referred to as 1+ in 5 patients. Finally, the CRP was significantly higher in patients with infection compared to patients without infection (p<0.001).

TABLE 1

| | Group A | Group B |
|---|---|---|
| Leukocyte esterase | 1/60 | 19/2* |
| (positive/negative) | | |
| Glucose (positive/negative) | 43/18 | 5/16** |
| CRP (mg/L) | 7.92 ± 20.0 | 40.3 ± 30.6*** |

*p < 0.001 vs Group A;
**p = 0.0016 vs Group A;
***p < 0.0005 vs Group A

The test of leukocyte esterase showed highest sensitivity and specificity values, followed in order by CRP and glucose as shown in Table 2 (sensitivity).

When the leukocyte esterase was combined with CRP, the sensitivity increased to 100%, while no differences were observed for leukocyte esterase combined with glucose. A sensitivity of 95.2% was observed when the CRP measures were combined with glucose test.

TABLE 2

| | Leukocyte | | |
|---|---|---|---|
| | esterase | Glucose | CRP |
| Sensitivity | 90.5% | 76.2% | 85.7% |
| | (68.2-98.3) | (52.4-90.9) | (62.6-96.2) |
| Specificity | 98.4% | 70.5% | 88.5% |
| | (90-0-99.9) | (57.2-81.1) | (79.2-95.9) |
| Positive predictive value | 95.0% | 47.0% | 72% |
| | (73.0-99.7) | (30.1-64.6) | (50.4-87.1) |
| Negative predictive value | 96.8% | 89.6% | 94.7% |
| | (87.8-99.4) | (76.6-96.1) | (84.4-98.6) |

In case of collected additional synovial fluid were available in the extraction syringe 24 and if necessary, collecting said synovial fluid in a totally sterile way and send it to a laboratory for culture analysis is possible. For this purpose, it is sufficient to close said third port 423 and to open said first 421 and second 422 port of said second three-way valve 42. Subsequently, the plunger of the syringe extraction 24 is pressed and, by means of said suction syringe 43, the synovial fluid previously collected in the reservoir of said extraction syringe 24 is aspirated. The path of the synovial fluid is that of the arrow C. Finally, said second port 422 of said second three-way valve 42 is closed and the plunger of the suction syringe 43 is pressed, so as to convey the synovial fluid collected in vacutainer 44, according to arrow D.

It is considered that typically 5-10% of the synovial fluid extracted presents traces of blood, that make the color of the same tending to red. In such situations it is commonly said that the color changes to red. This implies that the reading and the detection of the esterase reagent, which is based on a red coloration of the esterase, can be not accurate or not reliable.

To reduce this problem the extraction device comprises means for filtering the plasma 5. Said means for filtering the plasma 5 comprise a third three-way valve 51, having the first port 511, connected to the section of the extraction duct 21 connected to said third 233 port of said first three-way valve 23 and the third port 513 connected to the section of extraction duct 21 connected to said first port 421 of said second three-way valve 42.

Said means for filtering the plasma 5 (see FIG. 2) comprise a supplementary circuit, also comprising a filtering syringe 52, connected to said second port 512 of said third three-way valve 51. Said filtering syringe 52 is, in the present embodiment, preloaded with lysing.

Properly opening the ports of said first three-way valve 23 and of said third three-way valve 51 of said plasma filtering means 5, the extracted synovial fluid and preliminarily collected in said extraction syringe 24 is collected into said syringe filtering 52, so as to mix it with said lysing reagent, thereby obtaining the desired filtering. Said synovial fluid thus collected is then transmitted to said analysis device 3.

FIG. 3 shows a further embodiment of the extraction and analysis device 1, wherein the extraction line 2 comprises a first duct 211, which connects the withdrawal needle 22 and the analysis unit 3, and a second duct 212, which connects the extraction syringe 24 to the first duct 211.

Said first three-way valve 23 is arranged along said first duct 211 and on second port 232 presents the second duct 212. Along the second duct 212 a four-way valve 25 is arranged, which is substantially constituted by a common three-way valve, such as those described above, and a further port, which is not selectively closable on the port, so as to be also selectively openable or closable by means of its three-way valve. Said four-way valve 25 has the first port 251 connected to said second port 232 of said first three-way valve 23, i.e. to said first duct 211. The second port 252 connected to said vacutainer 44 and the third port 253 connected to said syringe extraction 24. The fourth port 254, not selectively closable, is connected to a filtering line 56, that will be better said below.

To make the ports of the four-way valve 25 selective, safety clamps 27 are arranged on the ducts in correspondence of the doors, closing or opening which, it is possible to close or to open the ports of the valve itself.

In correspondence of said extraction syringe 24 and of said filtering syringe 52, both a three-way valve, indicated respectively with 26 and 53, as well as a respective air filter 26′ and 53′ are installed, properly selectable by means of the ports of the three-way valves 26 and 53.

Said plasma filtering means 5 are arranged in series between said second port 232 of said first three-way valve 23 and said four-way valve 25.

Furthermore, said filtering means comprise a fourth three-way valve 54, having the first port 541 connected to said filtering syringe 52 and the second port 542 connected to said filtering line 56, which connects, by means of a three-way valve 57, to an analysis unit 3 and to a further three-way valve 58, 254 to said fourth port of said four-way valve 25.

The filtering means also comprise a filter for blood contamination 55, connected to the second port 512 of said third three-way valve 51 of the plasma filter means 5.

When the synovial fluid is collected in said extraction syringe 24, following the arrow A′, if necessary it is sent in said filtering syringe 52, which in this case can or cannot provide the preloaded lysing, according to the arrow B′, so as to pass through the filter for blood contamination 55. Subsequently, the synovial fluid is transmitted to said analysis device 32, according to the arrow C′, through filtering line 56, by the appropriate opening or closing of the valve ports 57 and 58 on the filtering line 56.

The synovial fluid present in the reservoir of the syringe 52 can then be re-collected in the vacutainer 44, flowing in the direction of arrow D′, closing the first 251 and the third port 253 and closing the clamp 27 on the third port 253, and opening the second port 252 and the safety clamp 27 on said fourth port 254 and said second port 252.

Also in this case, the synovial fluid filtered from the blood traces, when arranged in contact with the three reagents, allows to detect the degree of the infection by colorimetric enzymatic test.

In any case, once collected into the syringe 24, the synovial fluid may be subsequently inserted directly into the vacutainer 44, in case of the withdrawal were not entirely filtered or not filtered at all.

As it can be observed, the extraction and analysis device 1 is completely sterile and closed, in such a way that, once the synovial fluid is aspirated from the articulation, there is no of contamination of the liquid from the outside.

The use of clamps 27 allows the closure of the ducts on which they are arranged, by restriction. In particular, it is observed the clamp 27 arranged on the conduit 254 connected to the fourth port of said four-way valve 25, which allows the selective closure.

An advantage of the present invention is that of allowing a test on the synovial fluid pre-, intra- and post-operative, allowing in this way to obtain an immediate diagnosis (60-120 seconds) on the state of the articulation.

A further advantage of the present invention is to allow the real-time analysis of synovial fluid in a completely closed circuit way, allowing the withdrawal of the synovial fluid and its analysis in line, in such a way as to obtain a diagnosis in immediate times. The device is based on the use of different markers, such as leukocyte esterase and glucose, but it can be customizable by adding other markers according to the surveys to be carried out.

Another advantage of the present invention is that of allowing the possible collection of the synovial fluid by means of a vacuum system always, in line, in a test tube, vacutainer type or the like, which can be transported into the laboratory for cultural examination.

The present invention has been described for illustrative but not limitative purposes, according to its preferred embodiments, but it is to be understood that modifications and/or changes can be introduced by those skilled in the art without departing from the relevant scope as defined in the enclosed claims.

1. A device for extraction and analysis (1) of anatomical tissues, such as synovial liquid and the like, comprising
an extraction line (2, 21, 211, 212) of said tissue to be analyzed, having a needle (22), for the withdrawal of said tissue to be analyzed, an extraction duct (21, 211, 212), an extraction syringe (24), having an inlet/outlet mouth connected fluid-dynamically to said needle (22) by means of said extraction duct (21, 211, 212), and
an analysis device (3), fluid-dynamically connected to said inlet/outlet mouth of said extraction syringe (24) by means of said extraction duct (21, 211, 212), said analysis device (32) having one or more receptacles (321, 322, 323), in each of which a respective reagent is arranged, and reading means (33), for detecting possible alterations of said reagents,
said analysis device (3) also comprises a first receptacle (321), into which a first reagent is placed,
characterized in that said analysis device (3) comprises a second receptacle (322), into which a the second reagent is placed, and a third receptacle (323), into which a third reagent is placed, and
in that said extraction duct (21, 211, 212) of said extraction line (2) comprises a first three-way valve (23), having a first port (231), connected to said withdrawal needle (22), a second port (232), connected to said inlet/outlet mouth of said extraction syringe (24), and a third port (233) connectable to said analysis device (3), each of said ports (231, 232, 233) being selectively openable or closable independently of the other.

2. The device (1) according to claim 1, characterized in that said first reagent is esterases, said second reagent is CRP (C-Reactive Protein) and said third reagent is glucose.

3. The device (1) according to claim 1, characterized in that said reading means comprise a reading scale (33), capable to show the color variations of said reagents, allowing to detect the degree of infection of said anatomical tissue, as synovial liquid and the like.

4. The device (1) according to claim 1, characterized in that said extraction line (2) comprises air filtering means, in their turn comprising
an air filter (26′) and
a respective three-way valve (26), having three ports selectively openable or closable independently of the other, one of said three ports being connected to said inlet/outlet mouth of said extraction syringe (24), and another of said three ports being connected to said air filter (26′).

5. (canceled)

6. The device (1) according to claim 1, characterized in that it comprises plasma filtering means (5), adapted to filter the plasma from the said tissue to be analyzed, arranged between said inlet/outlet mouth of said extraction syringe (24) and said analysis device (3).

7. The device (1) according to claim 6, characterized
in that said plasma filtering means (5) comprise a filtering syringe (52), having an inlet/outlet mouth, a third three-way valve (51), having a first port (511), fluid-dynamically connected to said inlet/outlet mouth of said extraction syringe (24), a second port (512), to which the inlet/outlet mouth of said filtering syringe (52) is connected, and a third port (513), fluid-dynamically and selectively connected to said analysis device (3).

8. The device (1) according to claim 7, characterized
in that said extraction line (2) comprises a first duct (211), which connects said withdrawal needle (22) and the analysis unit (3), said first three-way valve (23) being arranged along said first duct (211), and a second duct (212), which connects the extraction syringe (24) to said first duct (211), a four-way valve (25) having a first port (251) fluid-dynamically connected to said withdrawal needle (22), a second port (252) fluid-dynamically connectable to said vacutainer (44), a third port (253) fluid-dynamically connectable to said extraction syringe (24) and a fourth port (254), each of said ports (251, 252, 253, 254) being selectively opened or closed independently of the other, and
in that said filtering means (5) comprises a filtering line (56), fluid-dynamically connected between said filtering syringe (52) and said analysis unit (3), and a further three-way valve (58), connected to said fourth port (254) of said four-way valve (25).

9. The device (1) according to claim 8, characterized in that it comprises closing means (26), as a clamp and the like, arranged in one or more of said ports (251, 252, 253, 254) of said four-way valve (25), said closing means (26) being preferably arranged on said first (251), second (252) and fourth (254) ports.

10. The device (1) according to claim 8, characterized in that said four-way valve comprises a three-way valve, whose ports (251, 252, 253) are selectively openable or closable independently of the other, and a fourth port (254).

11. The device (1) according to claim 6, characterized in that said filtering means comprise lysing, preloaded within said filtering syringe (52).

12. The device (1) according to claim 6, characterized in that said filtering means comprise a filter for blood contamination (55) interposed between said inlet/outlet mouth of said extraction syringe (24) and said inlet/outlet mouth of said filtering syringe (52).

13. The device (1) according to claim 1, characterized in that said device (1) also comprises an anatomical tissues collecting line (4), having a vacutainer (44), fluid-dynamically connected to the inlet/outlet mouth of a suction syringe (43).

14. The device (1) according to claim 13, characterized in that said collecting line comprises said suction syringe (43) having an inlet/outlet mouth fluid-dynamically connected to said inlet/outlet mouth of said extraction syringe (24).

15. The device (1) according to claim 13, characterized in that said collecting line (4) comprises a second three-way valve (42), having a first port (421), fluid-dynamically connected to said third port (233) of said first three-way valve (23) along said extraction duct (21, 211, 212) of said extraction line (2), a second port (422) fluid-dynamically connected to said inlet/outlet mouth of said suction syringe (43), and a third port (423), fluid-dynamically connected to said analysis device (3), each of said ports (421, 422, 423) being selectively openable or closable independently of the other.

16. The device (1) according to claim 15, characterized in that said collecting line (4) comprises filtering means comprising
an air filter and
a respective three-way valve, having three ports selectively openable or closable independently of the other, one of said three ports being connected to said inlet/outlet mouth of said suction syringe (43), and another one of said three ports being connected to said air filter.
